(19) United States
(12) Patent Application Publication
(10) Pub. No.: US 2009/0132002 A1
(43) Pub. Date: May 21, 2009

(54) BAROREFLEX ACTIVATION THERAPY WITH CONDITIONAL SHUT OFF

(75) Inventor: Robert S. Kieval, Medina, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Minneapolis, MN (US)

(21) Appl. No.: 12/116,435

(22) Filed: May 7, 2008

Related U.S. Application Data
(60) Provisional application No. 60/917,377, filed on May 11, 2007.

(51) Int. Cl. A61N 1/36

(52) U.S. Cl. 607/44; 607/62

(57) ABSTRACT

An exemplary embodiment of the present invention provides systems, devices, and methods for using the same for activating (stimulating) the baroreflex system of a patient using a baroreflex activation system which may be automatically shut off or discontinue therapy by sensing/monitoring/interpreting sensed data which is indicative of a physiological condition of a patient.

### CROSS-REFERENCES TO RELATED APPLICATIONS

The present application claims the benefit of provisional U.S. Application No. 60/917,377 (Attorney Docket No. 021433-003200US), filed May 11, 2007, the full disclosure of which is incorporated herein by reference. This application is also related to, but does not claim the benefit of the following U.S. patents and applications, all of which are fully incorporated herein by reference in their entirety: U.S. Pat. Nos. 6,522,926; 6,616,624; 6,985,774; 7,158,832; 6,850,801; PCT Patent Application No. PCT/US01/30249 filed Sep. 27, 2001 (Attorney Docket No. 021433-000140PC); U.S. patent application Ser. No. 10/284,063 (Attorney Docket No. 021433-000150US) filed Oct. 29, 2002; Ser. No. 10/453,678 (Attorney Docket No. 021433-000210US) filed Jun. 2, 2003; Ser. No. 10/402,911 (Attorney Docket No. 021433-000410US) filed Mar. 27, 2003; Ser. No. 10/402,393 (Attorney Docket No. 021433-000420US) filed Mar. 27, 2003; 60/549,760 (Attorney Docket No. 021433-001100US) filed Mar. 2, 2004; Ser. No. 10/818,738 (Attorney Docket No. 021433-000160US) filed Apr. 5, 2004; and 60/584,730 (Attorney Docket No. 021433-001200US) filed Jun. 30, 2004; Ser. No. 10/958,694 (Attorney Docket No. 021433-001600US) filed Oct. 4, 2004; Ser. No. 11/071,602 (Attorney Docket No. 021433-00110US) filed Mar. 2, 2005; Ser. No. 11/168,231 (Attorney Docket No. 021433-001210US) filed Jun. 27, 2005; 60/88,2478 (Attorney Docket No. 021433-002400US) filed Dec. 28, 2006; 60/883,721 (Attorney Docket No. 021433-002500US) filed Jan. 5, 2007; and 60/894,957 (Attorney Docket No. 021433-002600US) filed Mar. 15, 2007; the full disclosures, all of which are hereby incorporated by reference.

### BACKGROUND OF THE INVENTION

The present invention relates generally to medical devices and methods of use for the treatment and/or management of cardiovascular, neurological, and renal disorders, and more specifically to devices and methods for controlling the baroreflex system of a patient for the treatment and/or management of cardiovascular, neurological, and renal disorders and their underlying causes and conditions, more particularly to baroreflex systems and methods with smart processes for controlling therapy.

Hypertension, or high blood pressure, is a major cardiovascular disorder that is estimated to affect 65 million people in the United States alone, and is a leading cause of heart failure and stroke. It is listed as a primary or contributing cause of death in over 200,000 patients per year in the United States alone. Hypertension occurs in part when the body's smaller blood vessels (arterioles) constrict, causing an increase in blood pressure. Because the blood vessels constrict, the heart must work harder to maintain blood flow at the higher pressures. Sustained hypertension may eventually result in damage to multiple body organs, including the kidneys, brain, eyes, and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke.

Sustained high blood pressure may eventually result in an enlarged and damaged heart (hypertrophy), which may lead to heart failure. Heart failure is the final common expression of a variety of cardiovascular disorders, including ischemic heart disease. It is characterized by an inability of the heart to pump enough blood to meet the body's needs and results in fatigue, reduced exercise capacity and poor survival. Congestive heart failure (CHF) is an imbalance in pump function in which the heart fails to maintain the circulation of blood adequately. The most severe manifestation of CHF, pulmonary edema, develops when this imbalance causes an increase in lung fluid due to leakage from pulmonary capillaries into the lung. The most common cause of heart failure is coronary artery disease, which is secondary to loss of left ventricular muscle, ongoing ischemia, or decreased diastolic ventricular compliance. Other causes of CHF include hypertension, valvular heart disease, congenital heart disease, other cardiomyopathies, myocarditis, and infectious endocarditis.

A number of different treatment modalities may be attempted for treating heart failure, such as medications, mechanical restriction of the heart, surgical procedures to reduce the size of an expanded heart and the like.

Additionally, with the use of any methods including devices, the physiological conditions of a patient may change rapidly in response to internal and/or external conditions such that continued use of such devices, may cause significant harm to the patient. For example, any one of such devices may continue its operation in one or more modes even if such operation may be adverse to the patient's condition without proper safety measures.

Therefore, it would be desirable to provide improved methods and apparatus having smart processes for controlling their operation. Ideally, such methods and apparatus would be minimally invasive, with few if any significant side effects. Ideally, one or more underlying mechanisms causing heart failure could be treated in some cases. At least some of these objectives will be met by the present invention.

### BRIEF SUMMARY OF THE INVENTION

To address the problems of hypertension, heart failure, other cardiovascular disorders, nervous system and renal disorders, an exemplary embodiment of the present invention provides methods and devices (i.e., baroreflex activation device) for practicing the same, by which at least one baroreflex system within a patient's body is activated to achieve various therapeutic effects. In some exemplary embodiments, baroreflex activation therapy (BAT) suggests to the brain that the body is experiencing an increase in blood pressure. This suggestion may cause the brain to regulate (e.g., decrease) the level of sympathetic nervous system and neurohormonal activation. In some cases, the brain may also increase the level of sympathetic nervous system activity. These reactions may reduce blood pressure and have additional beneficial effects on the cardiovascular system and other body systems.

Methods and devices in accordance with some exemplary embodiments of the present invention may be used to activate baroreceptors, mechanoreceptors, pressoreceptors, or any other venous heart, or cardiopulmonary receptors which affect the blood pressure, nervous system activity, and neurohormonal activity in a manner analogous to baroreceptors in the arterial vasculation. For convenience, all such venous receptors (and/or nerves carrying signals from such receptors) may be collectively referred to herein as “baroreceptor/s.”

An exemplary embodiment of the present invention provides systems, devices, and methods allow for activating (stimulating) the baroreflex system of a patient using a baroreflex activation device which may automatically shut off or discontinue therapy by sensing/monitoring/interpreting sensed data which is indicative of a physiological condition of a patient. By way of example, the system may shut off therapy if the patient experiences a change in his/her condition where the continuation of the baroreflex therapy may be adverse to the patient's health.

It should be further understood by those skilled in the art, that the methods, devices, and systems according to exemplary embodiments of the present invention are further applicable to modify any one or more of the nervous system activity, autonomic nervous system activity, sympathetic/parasympathetic nervous system, or metabolic activity of the patient.

An exemplary embodiment of the present invention provides for the activation of the baroreflex system of a patient with a baroreflex activation device. A baroreflex activation therapy for a patient is normally determined and chosen by the healthcare provider. One or more parameters which are indicative of one or more physiological conditions of the patient are chosen and a threshold range for such parameter is selected. By way of example, and not limitation, the parameter may be the CO2 level in the blood of the patient. Other examples of such parameters include, but are not limited to: heart rate, blood pressure, ECG, oxygen saturation, blood pH, activity level (e.g., exercising, rest), prone posture, supine posture, core body temperature, respiration rate, and respiration depth, intracardiac pressure, timing of contractions of atria, and ventricles of the heart. In some embodiments, the one or more parameters are sensed by one or more sensors. The parameter may be sensed such that the system becomes aware of the value or condition of the parameter. In some embodiments, the parameter is sensed/monitored during a time period determined by the healthcare provider. The methods, embodying features of an exemplary embodiment of the present invention, modify/adjust the baroreflex therapy in response to the value of the monitored parameter. The therapy may be adjusted if the parameter value is outside of the threshold range. In some embodiments, the threshold range may comprise a lower value, which if the parameter falls below, the therapy discontinues. In some embodiments, the system may completely shut down its operation. In some embodiments, the adjusting may be discontinuation of the baroreflex activation therapy. In an embodiment, the method continues monitoring the parameter and comparing it to the threshold range to determine whether the baroreflex activation therapy should resume. In some embodiments, the baroreflex activation therapy continues as long as the value of the monitored parameter is greater than or equal to the threshold range. As used herein, the terms “sensed/sensing” and “monitor/monitoring” may be used interchangeably unless otherwise stated. In some embodiments, the threshold may be stored in a memory of the baroreflex activation device.

The therapy may operate in a closed loop or an open loop. By way of example, the discontinuation of the therapy may be through intervention by the patient/health care provider, or by way of algorithms which control the therapy and are programmed into the system.

In some embodiments, the therapy may be resumed by either or both the system itself and the patient/healthcare provider when the value of the parameter is no longer outside the parameter's threshold range (e.g., it is no longer below the lower limit of the range). By way of example, when the device or system provides for continued monitoring of the parameter, it may resume therapy once the parameter value reaches back within (or elevates above the lower value of) the threshold range.

In some embodiments, the baroreflex therapy comprises one or more therapy regimens, with the regimens delivering the baroreflex therapy at different doses/intensities such that the baroreflex system is activated (stimulated) to varying degrees. As used hereinafter, dose/intensity may be used to further describe some features of the invention. In some embodiments, the dose/intensity of the regimens may be changed by adjusting one or more characteristics of pulses generated by a pulse generator for activating the baroreflex activation device. Such characteristics include one or more of duty cycle, pulse amplitude, pulse width, pulse frequency, pulse separation, pulse waveform, pulse polarity, pulse shape, and pulse phase. By way of example, when the baroreflex therapy is discontinued, the dose/intensity will be zero. In some embodiments, methods embodying features of an exemplary embodiment of the present invention include establishing a target range for one or more parameters of interest.

In some embodiments, the one or more parameters are sensed by one or more sensors (further described below). The parameter may be sensed such that the system becomes aware of the value or condition of the parameter. In some embodiments, the parameter is sensed/monitored during a time period determined by the healthcare provider. The methods, embodying features of an exemplary embodiment of the present invention, modify/adjust the baroreflex therapy in response to a value of the monitored parameter. In some embodiments, the therapy is delivered at an initial dose/intensity. Upon sensing a change in the parameter, the method compares the value of the sensed parameter to the threshold range. In some embodiments, if the value of the parameter is outside the threshold range (e.g., below the threshold range), the method discontinues delivery of the therapy. If the value of the parameter is within the (e.g., greater than) threshold range, the value is then compared to the target range. The therapy may continue if the value of the parameter is within (e.g., at least equal to a lower limit of) the target range. In an embodiment, if the value of the parameter is less than the target range, therapy may change to a different therapy delivering a lower dose/intensity. If the value of the parameter is greater than the target range, therapy may change to a different therapy delivering a higher dose/intensity.

The system continues with delivery of therapy until such time that, either due to intervention by the patient/healthcare provider or the device/system, the therapy is ceased (e.g., according to the algorithms of the present method, or if the system runs out of energy).

In some embodiments as indicated above, the system may continue sensing/monitoring of the parameter/s. Once the value of the parameter is within the threshold range (e.g., or above the lower limit of the threshold range), the therapy may resume.

In an embodiment, the monitoring of the parameter is achieved by measuring at least one electrical potential difference within the body of the patient. In some exemplary embodiments, the monitoring of the parameter is accomplished by measuring a voltage difference between a first conductive element and a second conductive element of the baroreflex activation system. The first and second conductive elements include, respectively, a first and a second electrodes. The electrical potential difference may be repeatedly measured, and used to obtain a digitized electrocardiogram waveform. In one embodiment, the method includes identifying at least one R-wave in the electrocardiogram. The time interval between at least one pair of R-waves may also be measured. In an embodiment, the method includes identifying at least one R-wave peak, and it may also include measuring the time interval between at least one pair of such R-wave peaks.

In an exemplary embodiment, a system for treating a patient by providing baroreflex activation therapy to the patient is provided. The system, generally, includes a therapy circuitry for delivering baroreflex activation therapy to the patient, a controller circuitry connectable to the therapy circuitry and configured for controlling the baroreflex activation therapy to the patient, and a memory circuitry in communication with the controller and configured for storing information regarding the baroreflex activation therapy.

The baroreflex activation therapy may further include a plurality of therapy regimens with different intensity levels. A pulse generator configured for generating stimulation pulses to activate the baroreflex system of the patient may be included as part of the baroreflex activation device. The pulse generator is configured to deliver a plurality of pulses having different intensity levels. One of such intensity levels is at or close to zero. The baroreflex activation therapy device, typically, further includes at least one electrode assembly which is generally locatable proximate one or more baroreceptors of the patient.

The system may further include a monitoring circuitry which is connectable to the controller circuitry. The system may further comprise a sensor connectable to the monitoring circuitry and which is configured for sensing a patient parameter which is indicative of a physiological condition of the patient. The sensor may include one or more of suitable sensors such as extracardiac, electrocardiogram, intracardiogram, pressure sensor, and accelerometer, or any of the other sensors mentioned earlier. The controller circuitry is configured to adjust the baroreflex activation therapy based on information received by way of the sensor. The system may further include a switching circuitry which is connectable to the monitoring circuitry and the therapy circuitry, for adjusting the baroreflex activation therapy based on the information received from the monitoring circuitry and the therapy circuitry. The switching circuitry, typically, is connectable to at least one of the electrode assembly locatable proximate one or more baroreceptors of the patient.

The system may be housed within a single conductive housing; and may furthermore, be implantable in the patient. The system may further be configured for communication with other devices such as cardiac rhythm management devices including cardiac resynchronization therapies such as pacemakers and combination pacemaker/defibrillators.

In another exemplary embodiment, a system for treating a patient, such as one for providing baroreflex activation therapy includes a therapy circuitry for providing baroreflex activation therapy to a body of the patient, a monitoring circuitry that is capable of measuring a biopotential within the body of the patient for producing an electrocardiogram signal, a switching circuitry coupled to the therapy circuitry and the measurement circuitry, and a control circuitry which is coupled to the switching circuitry. The control circuitry is configured for directing the switching circuitry to periodically connect one or more electrodes which are locatable at or near a baroreceptor of the patient, to the therapy circuitry for providing baroreflex activation therapy to the body of the patient. The control circuitry is further configured to direct the switching circuitry to periodically connect the one or more electrodes to the monitoring circuitry for measuring the biopotential within the body of the patient for producing the electrocardiogram signal.

In another exemplary embodiment, a baroreflex activation therapy system is provided, including a therapy circuitry which is connected to a first therapy terminal and a second therapy terminal for producing a baroreflex activation therapy signal, a measurement circuitry that is capable of measuring a voltage between a first measurement terminal and a second measurement terminal, a switching circuitry connected to the first measurement and second measurement terminals as well as the first and the second therapy terminals. The switching circuitry is configured to selectively couple the first electrode and second electrode assemblies, which are locatable at or near the patient's baroreceptors, respectively, for providing baroreflex activation therapy to the body of the patient.

The switching circuitry may selectively couple one or more of electrodes of an electrode assembly, to one or more of the measurement terminals, for measuring electric potential difference with the body of the patient.

For example, the switching circuitry may selectively couple the first electrode and a third electrode to the first and second measurement terminals, respectively, for measuring an electric potential difference within the body of the patient. One or more of the electrodes, such as the first electrode, may include an inner electrode, and optionally one or more outer electrodes.

The baroreflex activation therapy electrode assembly/assemblies may include a sheet of flexible material and a plurality of electrodes secured over a first surface of the sheet. The one or more electrodes may each have a proximal and a distal end, with the inner electrode when more than one outer electrode is present, located between the two outer electrodes. The electrodes may be so positioned on the flexible sheet such that the proximal end of the inner electrode is proximate the distal ends of the outer electrodes, while the distal end of the inner electrode is positioned proximate the proximal ends of the outer electrodes (or vice versa).

The switching circuitry may selectively couple the first electrode and a conductive housing of the baroreflex activation therapy system to the first and second measurement terminals, respectively, for measuring an electric potential difference with the body of the patient. The conductive housing, may be a hermatically sealed housing defining an interior. The therapy circuitry, the measurement circuitry, and the switching circuitry, may all be disposed in the interior of the housing.

In another exemplary embodiment, a baroreflex activation therapy device includes a memory storing a threshold value associated with a physiological parameter of a patient, a therapy circuitry for delivering baroreflex activation therapy to a body of the patient, a sensor for measuring a value of the physiological parameter of the patient, and a disable circuitry that disables the therapy circuitry if the measured value is below the threshold value. The system may further include any suitable sensor, such as a pressure sensor. The sensor may include a measurement circuitry connected to one or more electrodes for measuring a biopotential in the body of the patient.

In an exemplary embodiment, a baroreflex activation therapy system includes a therapy circuitry connected to a first and a second therapy terminal for producing a baroreflex activation therapy signal, a sensor connected to a first and a second therapy terminal, and a switching circuitry connected to the first and second sensor terminals as well as the first and the second therapy terminals. The switching circuitry selectively couples first and second electrodes to the first and second therapy terminals, respectively, for providing baroreflex activation therapy to the body of a patient. The sensor may include any suitable sensor such as a pressure sensor.

The switching circuitry may selectively couple any one or more electrodes, such as a first and a third electrode (of any one or more electrode assemblies) which are locatable at or near a baroreceptor of a patient, to any one or more of the sensor terminals such as the first and second terminals, respectively, for measuring an electric potential difference within the body of the patient.

One or more of the electrodes, such as the first electrode, may include an inner electrode, and optionally, one or more outer electrodes. The baroreflex activation therapy electrode assembly/assemblies may include a sheet of flexible material and a plurality of electrodes secured over a first surface of the sheet. The plurality of the electrodes may each have a proximal and a distal end, with the inner electrode when more than one outer electrode is present, located between the outer electrodes. A first lead may be electrically connected to the proximal ends of the first and second outer electrodes, with a second lead electrically connected to the proximal end of the inner electrode. The electrodes may be so positioned on the flexible sheet such that the proximal end of the inner electrode is proximate the distal ends of the outer electrodes, while the distal end of the inner electrode is positioned proximate the proximal ends of the outer electrodes (or vice versa). The switching circuitry may selectively couple the first electrode and a conductive housing of a baroreflex activation therapy system to first and second sensor terminals, respectively. The conductive housing may be a hermatically sealed housing defining an interior, in which, the therapy circuitry and the switching circuitry are both housed.

In various embodiments, a control system may be used to generate a control signal which activates, deactivates, or otherwise modulates the baroreflex activation device. The control system may operate in an open-loop or a closed-loop mode. For example, in the open-loop mode, the patient and/or physician may directly or remotely interface with the control system to prescribe the control signal. In the closed-loop mode, the control signal may be responsive to feedback from a sensor, wherein the response is dictated by a preset or programmable algorithm defining a stimulus/activation therapy and the plurality of regimen. The stimulus (activation) therapy is preferably selected to promote long term efficacy and to minimize power requirements. It is theorized that uninterrupted baroreflex activation may result in the baroreflex and/or central nervous system becoming less responsive over time, thereby diminishing the effectiveness of the therapy. Therefore, the stimulus therapy may be selected to modulate the baroreflex activation device in such a way that the baroreflex maintains its responsiveness over time. Specific examples of stimulus regimens which promote long term efficacy are described in the applications earlier above incorporated herein by reference in their entirety.

Generally, any of a number of suitable anatomical structures may be activated to provide baroreflex activation. For example, in various embodiments, activating the baroreflex system may involve activating one or more baroreceptors, one or more nerves coupled with a baroreceptor, a carotid sinus nerve, or some combination thereof. In embodiments where one or more baroreceptors are activated, the baroreceptor(s) may sometimes be located in arterial vasculature, such as but not limited to a carotid sinus, aortic arch, heart, common carotid artery, subclavian artery, pulmonary artery, femoral artery and/or brachiocephalic artery. Alternatively, a baroreflex activation device may be positioned in the low-pressure side of the heart or vasculature, as described in U.S. patent application Ser. No. 10/284,063, previously incorporated by reference, in locations such as an inferior vena cava, superior vena cava, portal vein, jugular vein, subclavian vein, iliac vein, azygous vein, pulmonary vein and/or femoral vein. The baroreflex activation may be achieved, in various embodiments, by electrical activation, mechanical activation, thermal activation, biological activation, and/or chemical activation. Furthermore, baroreflex activation may be continuous, pulsed, periodic or some combination thereof, in various embodiments.

As suggested above, various embodiments of the inventive devices may be entirely intravascular, entirely extravascular, or partially intravascular and partially extravascular. Furthermore, devices may reside wholly in or on arterial vasculature, wholly in or on venous vasculature, or in or on some combination of both. In some embodiments, for example, implantable devices may be positioned within an artery or vein, while in other embodiments devices may be placed extravascularly, on the outside of an artery or vein. In yet other embodiments, one or more components of a device, such as electrodes, a controller or both, may be positioned outside the patient's body. In introducing and placing devices of an exemplary embodiment of the present invention, any suitable technique and access route may be employed. For example, in some embodiments an open surgical procedure may be used to place an implantable device. Alternatively, an implantable device may be placed within an artery or vein via a transvascular, intravenous approach. In still other embodiments, an implantable device may be introduced into vasculature via minimally invasive means, advanced to a treatment position through the vasculature, and then advanced outside the vasculature for placement on the outside of an artery or vein. For example, an implantable device may be introduced into and advanced through the venous vasculature, made to exit the wall of a vein, and placed at an extravascular site on an artery.

These and other aspects and embodiments of an exemplary embodiment of the present invention are described in further detail below, with reference to the attached drawings.

### BRIEF DESCRIPTION OF THE DRAWINGS

FIG. 1 is a schematic illustration of the upper torso of a human body showing the major arteries and veins and associated anatomy.

FIG. 2A is a cross sectional schematic illustration of a carotid sinus and baroreceptors within a vascular wall.

FIG. 2B is a schematic illustration of baroreceptors within a vascular wall and the baroreflex system.

FIG. 3A a block diagram of a baroreflex activation therapy system, embodying features of an exemplary embodiment of the present invention.

FIG. 3B a block diagram of another baroreflex activation therapy system, embodying features of an exemplary embodiment of the present invention.

FIG. 4A is a flow diagram of another process, embodying features of an exemplary embodiment of the present invention, for conditional shut off of baroreflex therapy.

FIG. 4B is a flow diagram of another process, embodying features of an exemplary embodiment of the present invention, for conditional shut off and restart of baroreflex therapy.

FIG. 4C is a flow diagram of another process, embodying features of an exemplary embodiment of the present invention, for conditional shut off and restart of baroreflex therapy.

FIG. 4D is a flow diagram of the process of FIG. 4C, embodying features of an exemplary embodiment of the present invention, for conditional shut off and restart of baroreflex therapy.

FIG. 5A is a graphical representation of the relationship between arterial pressure and the disabling of baroreflex therapy.

FIG. 5B is a graphical representation of the relationship between heart rate and the disabling of baroreflex therapy.

FIG. 6 is a flow diagram of a baroreflex activation system, embodying features of an exemplary embodiment of the present invention.

FIG. 7 is a block diagram of baroreflex activation system, embodying features of an exemplary embodiment of the present invention.

FIG. 8 is a block diagram of another baroreflex activation system, embodying features of an exemplary embodiment of the present invention.

### DETAILED DESCRIPTION OF THE INVENTION

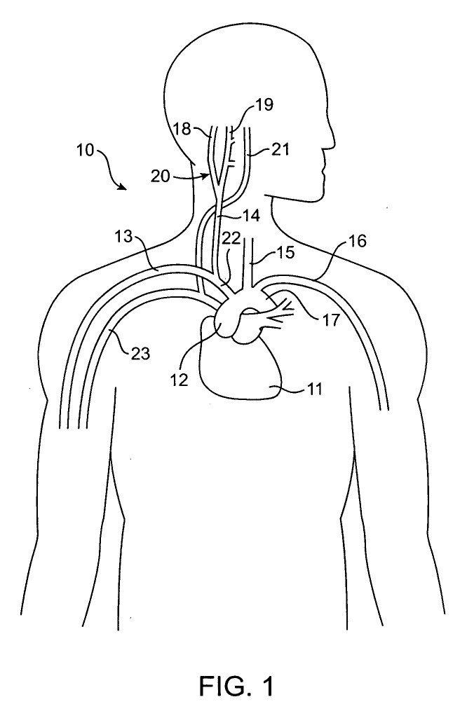

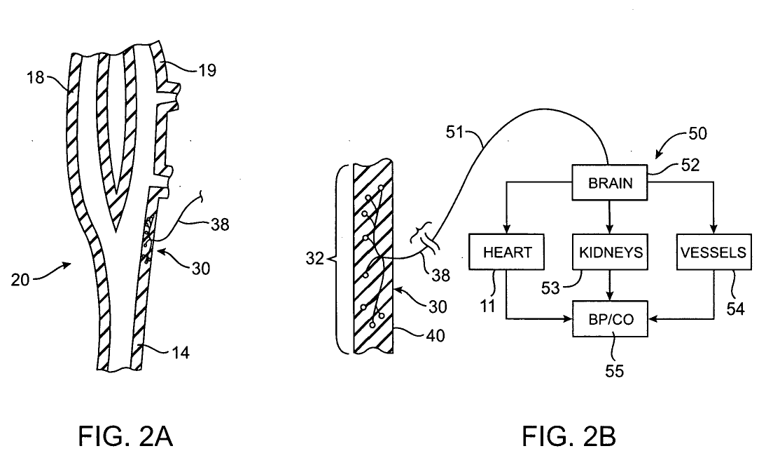

Referring now to FIGS. 1, 2A, and 2B, within the arterial walls of the aortic arch 12, common carotid arteries 14/15 (near the right carotid sinus 20 and left carotid sinus), subclavian arteries 13/16, and brachiocephalic artery 22, baroreceptors 30 are shown. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Baroreceptors 30 located in the right carotid sinus 20, the left carotid sinus, and the aortic arch 12 play the most significant role in sensing blood pressure that affects baroreflex system 50, which is described in more detail with reference to FIG. 2B.

With reference now to FIG. 2B, a schematic illustration shows baroreceptors 30 disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2B are primarily schematic for purposes of illustration.

In addition to baroreceptors, other nervous system tissues are capable of inducing baroreflex activation. For example, baroreflex activation may be achieved in various embodiments by activating one or more baroreceptors, one or more nerves coupled with one or more baroreceptors, a carotid sinus nerve or some combination thereof. Therefore, the phrase “baroreflex activation” generally refers to activation of the baroreflex system by any means, and is not limited to directly activating baroreceptor(s). Although the following description often focuses on baroreflex activation/stimulation and induction of baroreceptor signals, various exemplary embodiments of the present invention may alternatively achieve baroreflex activation by activating any other suitable tissue or structure.

Baroreflex signals are used to activate a number of body systems which collectively may be referred to as baroreflex system 50. Baroreceptors 30 are connected to the brain 52 via the nervous system 51, which then activates a number of body systems, including the heart 11, kidneys 53, vessels 54, and other organs/tissues via neurohormonal activity. Although such activation of baroreflex system 50 has been the subject of other patent applications by the inventors of the present invention, the focus of exemplary embodiments of the present invention is baroreflex system and methods using the same which allow for automatic shut off in certain conditions to prevent or minimize adverse effects of baroreflex activation on the brain 52.

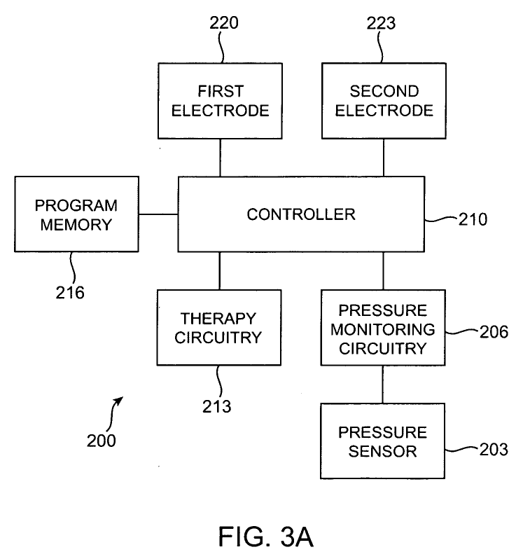

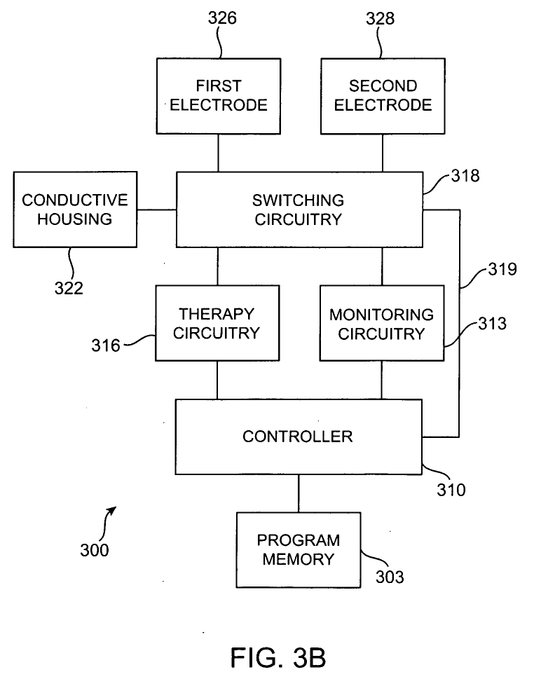

With reference to FIG. 3A, in an embodiment, a system 200 for activation/stimulation of the baroreflex system of a patient is shown. The system 200 includes a pressure sensor 203, connected to a pressure monitoring circuitry 206, which in turn is connected to a controller 210. A therapy circuitry 213 is also connected to the controller 210. A memory 216 is connected to the controller and provides the instruction and therapy algorithm to the controller 210. Electrode assemblies 220 and 223 are connected at one end to the controller, and at another end to the patient to provide baroreflex stimulation to the patient's baroreflex system. Each of the electrode assemblies 220 and 223 may comprise one or more electrodes suitable for delivering baroreflex activation therapy.

Now referring to FIG. 3B, an embodiment similar in some aspects to that shown in FIG. 3A, for activation/stimulation of the baroreflex system of a patient is shown. A memory 303 is connected to a controller 310 which is connected to a monitoring circuitry 313 and a therapy circuitry 316. Switching circuitry 318 is coupled to the controller by way of cable 319 It will be understood that cable 319 may include any number of conductors. Controller 31 may deliver control signals to switching circuitry 318 via the conductors of cable 319. The switching circuitry 318 is also electrically connected to a conductive housing 322. Electrode assemblies 326 and 328 are connected to the switching circuitry 318. In the embodiment of FIG. 3B, therapy circuitry 316 is connected to switching circuitry 318. Switching circuitry 318 is capable of selectively connecting electrode assemblies 326 and 328 to therapy circuitry 316 for providing baroreflex activation therapy (BAT) to the patient's baroreflex system. Also in the embodiment of FIG. 3B, monitoring circuitry 313 is connected to switching circuitry 318. Switching circuitry 318 is capable of selectively connecting electrode assemblies 326 and 328 to monitoring circuitry 313 for measuring electrical potential differences within the body of the patient. Controller 310 is connected to both therapy circuitry 316 and monitoring circuitry 313. Accordingly, controller 310 may deliver control signals to therapy circuitry 316 and monitoring circuitry 313. Controller

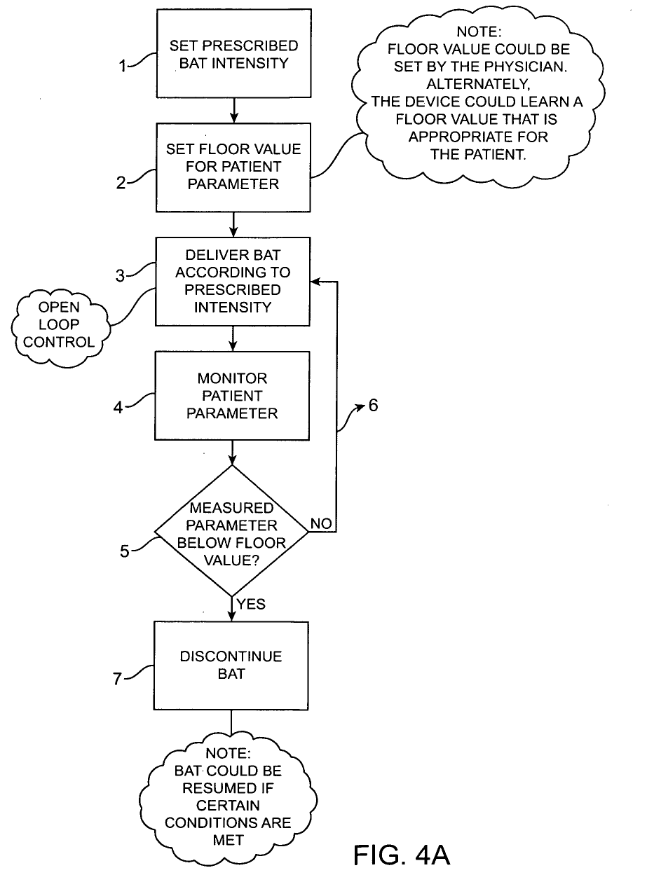

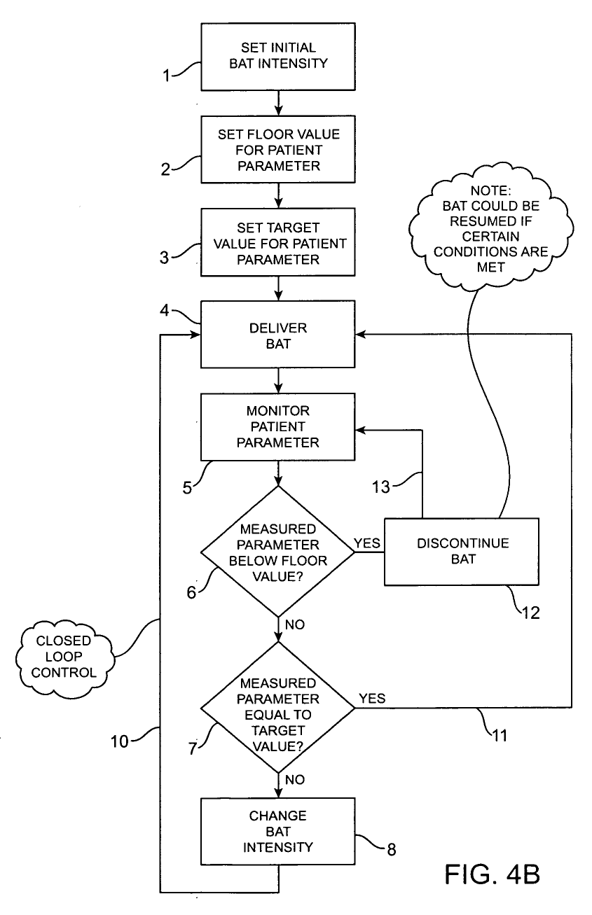

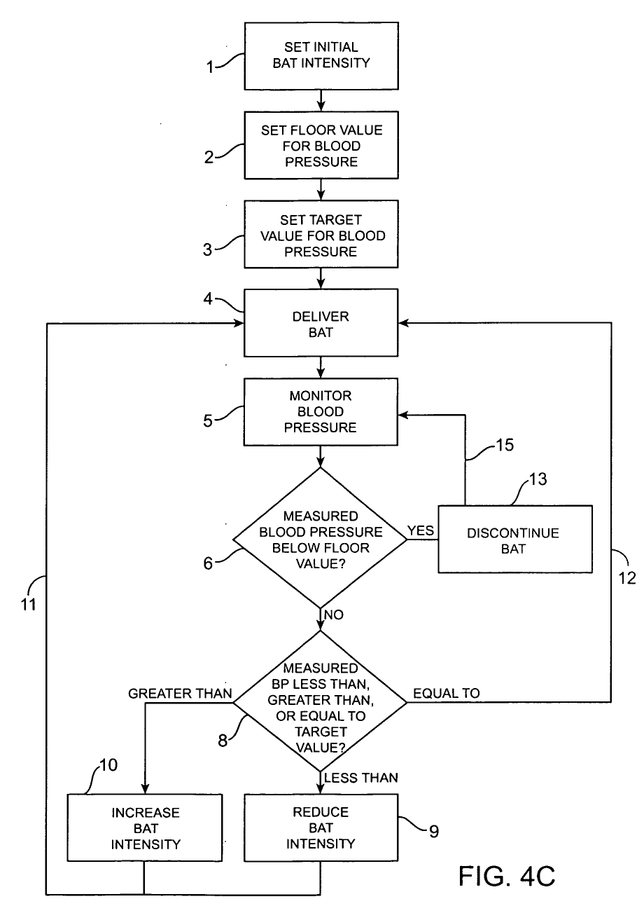

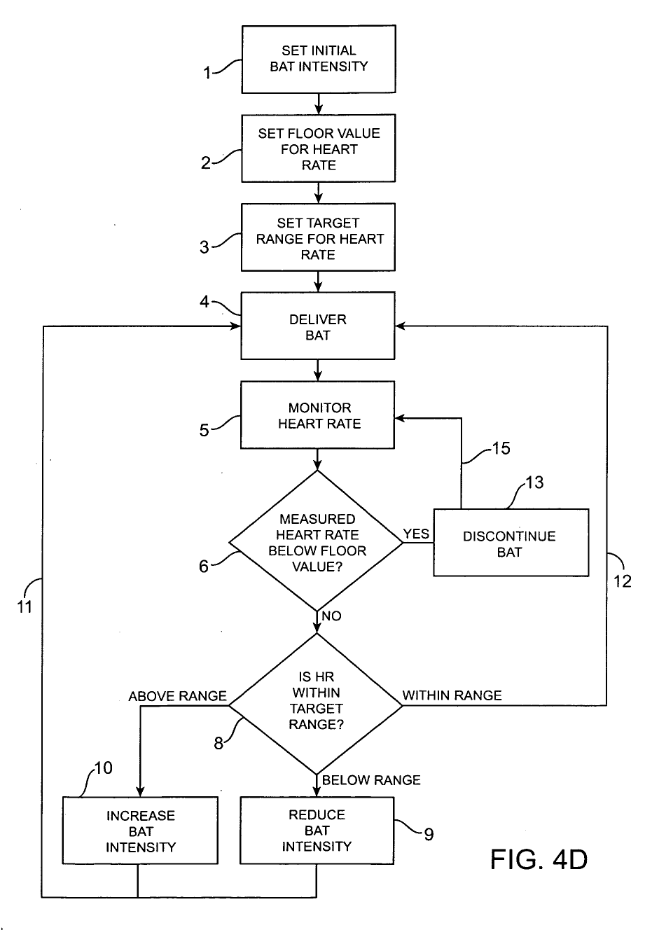

Now referring to FIG. 4A, a block diagram illustrates a method embodying features of an exemplary embodiment of the present invention. As shown, a prescribed baroreflex activation therapy (e.g., BAT intensity) is set by a medical care provider (e.g., a physician) (box 1). A threshold value (or range) is established for one or more parameters, indicative of a physiological condition, for a given patient (box 2). Alternatively, the system itself may learn a threshold value that is appropriate for the patient. It should be understood that, in some embodiments, the threshold value may be a range, an upper limit, a lower limit, or any combination thereof. The baroreflex activation therapy is delivered to the patient according to the prescribed intensity (box 3). The parameter of interest for the patient (see box 2) is monitored (box 4) during the baroreflex activation therapy and its value is compared with the established threshold value (box 5). If the value of the parameter is determined to be above the threshold value (box 5), the baroreflex activation therapy continues (line 6) and the cycle restarts back from box 3. If, however, the value of the parameter is determined to be outside of the threshold value, the baroreflex activation therapy is discontinued (box 7). In the process illustrated in FIG. 4A, baroreflex activation therapy is delivered according to a pre-determined prescribed therapy regimen such that the baroreflex system is activated with a pre-determined energy. If, however, the measured parameter drops below the threshold value, then the baroreflex activation therapy is discontinued (box 7).

Now referring to FIG. 4B, a block diagram illustrates a method embodying features of an exemplary embodiment of the present invention. As shown, a prescribed baroreflex activation therapy is set by a care provider (e.g., a physician) (box 1). The care provider may also set a threshold value (or range) established for one or more parameters of a given patient (box 2). As shown, the threshold value is established as a floor/lower limit, however, it should be understood that embodiments are possible in which the threshold value is a range, an upper limit, a lower limit, or any combination thereof. A target value (or range) for one or more parameters for a given patient may also be set by the care provider (box 3). Baroreflex activation therapy is delivered to the patient according to the prescribed intensity (box 4). The parameter of interest for the patient (see box 2) is monitored (box 5) during the baroreflex activation therapy and its value is compared with the established threshold value (box 6). If the value of the parameter is determined to be above the threshold value (box 6), the baroreflex activation therapy continues to the next step and it is determined whether the value of the parameter is equal to the target value for that parameter (box 7). If the measured (or calculated parameter) is not equal to the target value, the intensity of the baroreflex activation therapy is changed (box 8) and the cycle restarts (line 10) back from box 4.

If, however, earlier in the process (at box 7), it is determined that the value of the parameter is equal to the target value/range, the cycle returns to box 4 and baroreflex activation therapy continues according the prescribed intensity as initially set for the baroreflex activation therapy. If, however, during box 6 it is determined that the value of the measured (or calculated) parameter is below the threshold value, the baroreflex activation therapy is discontinued (box 12). In some exemplary embodiments, the system may be programmed to monitor (line 13) the parameter and if the parameter ever reaches above threshold level, the system may resume baroreflex activation therapy.

Now referring to FIG. 4C, a block diagram illustrates a method embodying features of an exemplary embodiment of the present invention. As shown, a prescribed baroreflex activation therapy is set by a caregiver (e.g., a physician) (box 1). The exemplary method illustrated in FIG. 4C also includes the step of setting threshold value for one or more parameters indicative of the patient's physiological condition (box 2). It should be understood that, in some embodiments, the threshold value may be a range, an upper limit, a lower limit, or any combination thereof. In the exemplary embodiment of FIG. 4C, blood pressure is the parameter of choice (box 2). A target value (or range) for patient blood pressure is established at box 3. The baroreflex activation therapy is delivered to the patient according to the prescribed intensity (box 4). The blood pressure is monitored (box 5) during the baroreflex activation therapy and its value is compared with the established threshold value (box 6). If the value of the blood pressure is determined to be above the threshold value (box 6), the baroreflex activation therapy continues to the next step and it is determined whether the blood pressure is less than, greater than, or equal to the target value for blood pressure (box 8). If the measured (or calculated) value for the blood pressure is not equal to the target value, the intensity of the baroreflex activation therapy is changed depending on whether the blood pressure value is less or greater than the target value (box 8). If the blood pressure value is less than the target value, the intensity of the baroreflex activation therapy is reduced (box 9) and if the blood pressure value is greater than the target value, the intensity of the baroreflex activation therapy is increased (box 10). After either box 9 or box 10, the cycle restarts (line 11) back to box 4.

If, however, earlier in the process (at box 8), it is determined that the value of the blood pressure is equal to the target value/range, the cycle restarts back (line 12) from (box 4) and baroreflex activation therapy continues according to the prescribed initially set intensity for the baroreflex activation therapy. If, however, during the step shown in box 6, it is determined that the blood pressure is below the threshold value, the baroreflex activation therapy is discontinued (box 13). In one embodiment, the system may be programmed to monitor the blood pressure (line 15) and if the blood pressure ever reaches a value above the threshold level, the system moves forward to the next step (box 6). This flow chart represents a process embodying features of an exemplary embodiment of the present invention, where the system automatically makes changes in BAT intensity.

Now referring to FIG. 4D, a block diagram illustrates a method embodying features of an exemplary embodiment of the present invention. As shown, a prescribed baroreflex activation therapy is set by a healthcare provider (e.g., a physician) (box 1). The healthcare provider may also set a threshold value (or range) for one or more parameters indicative of a physiological condition of the patient being treated (box 2). It should be understood that, in some embodiments, the threshold value may be a range, an upper limit, a lower limit, or any combination thereof. In the exemplary embodiment of FIG. 4D, heart rate is the physiological parameter illustrated in the flow chart (box 2). A target value (or range) is established for the patient's heart rate (box 3). The baroreflex activation therapy is delivered to the patient according to the prescribed intensity (box 4). The heart rate is monitored (box 5) during the baroreflex activation therapy, and its value is compared with the established threshold value (box 6). If the value of the heart rate is determined to be above the threshold value (box 6), the baroreflex activation therapy continues to the next step and it is determined whether the heart rate is within a target range for heart rate (box 8). If the measured (or calculated) value for the heart rate is not within the target range, the intensity of the baroreflex activation therapy is changed depending on whether the heart rate value is less or greater than the target value (box 8). If the heart rate value is less than the target value, the intensity of the baroreflex activation therapy is reduced (box 9) and if the heart rate value is greater than the target value, the intensity of the baroreflex activation therapy is increased (box 10). After either of box 9 or box 10, the cycle returns (line 11) to box 4.

If, however, earlier in the process (at box 8), it is determined that the value of the heart rate is determined to be within the target range, the cycle returns (line 12) to step 4 with no change in the intensity of the baroreflex activation. If, however, during box 6 it is determined that the heart rate is below the threshold value, the baroreflex activation therapy is discontinued (box 13). In some exemplary embodiments, the system may be programmed to continue monitoring the heart rate after the delivery of baroreflex activation therapy has been discontinued (line 15). If the heart rate exceeds the threshold level, then baroreflex activation therapy may be applied to the patient (box 6). This flow chart illustrates an exemplary embodiment of the present invention in which the system makes automatic adjustments to baroreflex activation therapy intensity

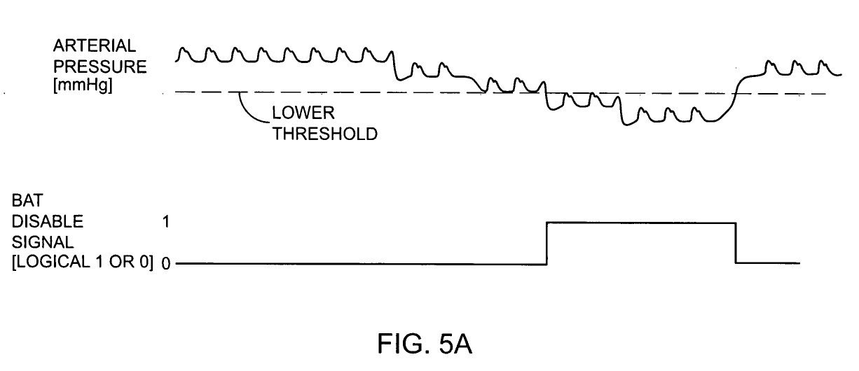

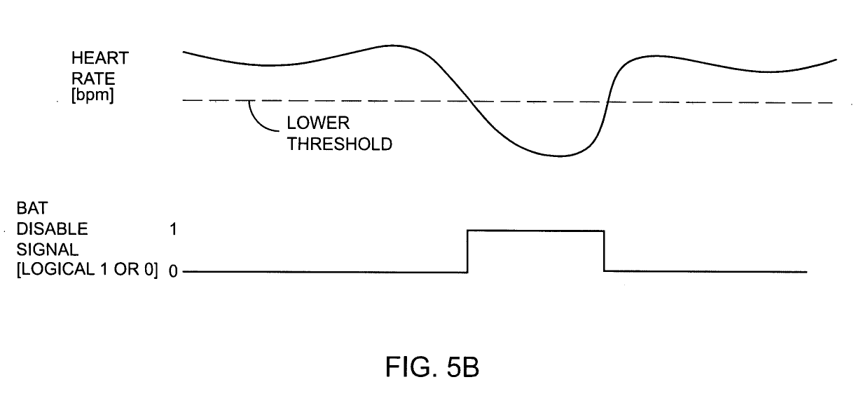

Now referring to FIG. 5A, a timing diagram illustrating the relationship between the arterial pressure (as the sensed/measured/calculated parameter) and the discontinuing of the baroreflex activation therapy is shown. An arterial pressure waveform is shown in FIG. 5A. When the arterial pressure falls below a threshold value, the measuring/monitoring portion of the baroreflex activation therapy system provides a signal to the therapy delivery portion of the baroreflex activation therapy system to discontinue baroreflex activation therapy. In the exemplary embodiment of FIG. 5A, this signal as shown as a logical signal having logical value of either 1 or 0. This logical signal may be provided to a controller (e.g., a microprocessor). When the signal has a logical value of 1, the controller discontinues baroreflex activation therapy.

Now referring to FIG. 5B, a timing diagram illustrating the relationship between the heart rate (as the sensed/measured/calculated parameter) and the discontinuing of the baroreflex activation therapy is shown. As shown in FIG. 5B, when the heart rate falls below a threshold value, the system provides a signal to the system to discontinue baroreflex activation therapy. In the exemplary embodiment of FIG. 5B, this signal as shown as a logical signal having logical value of either 1 or 0. This logical signal may be provided to a controller (e.g., a microprocessor). When the signal has a logical value of 1, the controller discontinues baroreflex activation therapy.

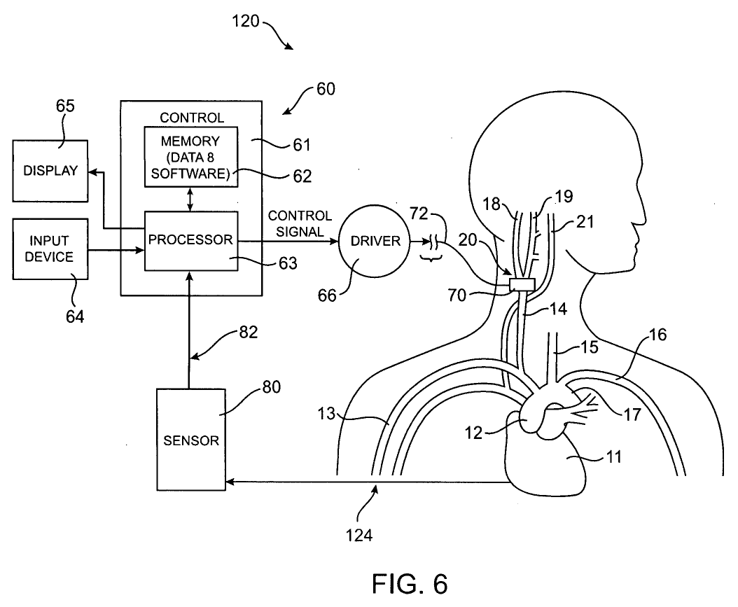

Now referring to FIG. 6, the general features of a baroreflex activation system usable in the practice of an exemplary embodiment of the present invention and incorporating one or more features of an exemplary embodiment of the present invention is shown. The system 120 includes a processor 63, a baroreflex activation device 70, and a sensor 80. For clarity, the sensor 80 is shown as one unit located outside the patient, such as would be the case if the sensor 80 comprised an external electrocardiogram (ECG) device. In alternative embodiments, however, the sensor 80 (or multiple sensors) may be located on or in the heart 11 or in any other suitable location within the patient. Optionally, processor 63 may be part of a control system 60, which may include a control block 61 (housing processor 63 and memory 62), a display 65 and/or and input device 64. Processor 63 is coupled with sensor 80 by an electric sensor cable or lead 82 and to baroreflex activation device 70 by an electric control cable 72. In alternative embodiments, lead 82 may be any suitable corded or remote connection means, such as a remote signaling device. Thus, processor 63 receives a sensor signal from sensor 80 by way of sensor lead 82 and transmits a control signal to baroreflex activation device 70 by way of control cable 72. In an alternative embodiment, the processor 63 may be combined in one unitary device with the baroreflex activation device 70.

As discussed above, in one embodiment, generally, the heart 11 may be coupled with the sensor 80 by way of one or more leads 124, such as with an ECG device. In other embodiments, the sensor(s) 80 may be attached directly to a wall of the heart 11 or to any other suitable anatomical structure.

As mentioned above, the sensor 80 generally senses and/or monitors one or more parameters, such as but not limited to change in heart rate, change in cardiac pressure(s), change in contraction timing of one or both atria and ventricles of the heart, change in electrocardiogram shape (such as T-wave shape), change in blood pressure and/or the like. The parameter sensed by sensor 80 is then transmitted to processor 63, which may generate a control signal as a function of the received sensor signal. A control signal will typically be generated, for example, when a sensor signal is determined to be indicative of physiological condition of the patient. If decreased cardiac efficiency, for example, is determined to be an advance indicator of the onset of heart failure, data that is sensed and processed and determined to be indicative of decreased efficiency will cause processor 63 to generate a control signal. The control signal activates, deactivates, modifies the intensity or timing of, or otherwise modulates baroreflex activation device 70. In some embodiments, for example, baroreflex activation device 70 may activate an ongoing baroreflex at a constant rate until it receives a control signal, which may cause the device 70 to either increase or decrease intensity of its baroreflex activation. In another embodiment, baroreflex activation device 70 may remain in a turned-off mode until activated by a control signal from processor 63. In another embodiment, when sensor 80 detects a parameter indicative of normal body function (e.g., steady heart rate and/or steady intracardiac pressures), processor 63 generates a control signal to modulate (e.g., deactivate) baroreflex activation device 70. Any suitable combination is contemplated in various embodiments.

Again, sensor 80 may comprise any suitable device that measures or monitors a parameter indicative of the need to modify baroreflex activation. For example, sensor 80 may comprise a physiologic transducer or gauge that measures cardiac activity, such as an ECG, or any other physiologic activity described above. Alternatively, sensor 80 may measure cardiac activity by any other technique, such as by measuring changes in intracardiac pressures or the like. Examples of suitable transducers or gauges for sensor 80 include ECG electrodes and the like. Although only one sensor 80 is shown, multiple sensors of the same or different type at the same or different locations may be utilized. Sensor 80 is preferably positioned on or near the patient's heart, on or near major vascular structures such as the thoracic aorta, or in another suitable location to measure cardiac activity, such as increased heart rate or pressure changes. Sensor 80 may be disposed either inside or outside the body in various embodiments, depending on the type of transducer or gauge utilized. Sensor 80 may be separate from baroreflex activation device 70, as shown schematically in FIG. 6, or may alternatively be combined therewith in one device.

The baroreflex activation component of the baroreflex activation device 70 may comprise a wide variety of devices which utilize mechanical, electrical, thermal, chemical, biological, or other means to activate baroreceptors 30 and/or other tissues. In many embodiments, particularly the mechanical activation embodiments, the baroreflex activation device 70 indirectly activates one or more baroreceptors 30 by stretching or otherwise deforming the vascular wall 40 surrounding baroreceptors 30. In some other instances, particularly the non-mechanical activation embodiments, baroreflex activation device 70 may directly activate one or more baroreceptors 30 by changing the electrical, thermal or chemical environment or potential across baroreceptors 30. It is also possible that changing the electrical, thermal or chemical potential across the tissue surrounding baroreceptors 30 may cause the surrounding tissue to stretch or otherwise deform, thus mechanically activating baroreceptors 30. In other instances, particularly the biological activation embodiments, a change in the function or sensitivity of baroreceptors 30 may be induced by changing the biological activity in baroreceptors 30 and altering their intracellular makeup and function.

Many embodiments of the baroreflex activation device 70 are suitable for implantation, and are preferably implanted using a minimally invasive percutaneous translumenal approach and/or a minimally invasive surgical approach, depending on whether the device 70 is disposed intravascularly, extravascularly, or within the vascular wall 40. The baroreflex activation device 70 may be positioned at any location where baroreceptors 30 which affect the baroreflex system 50 are numerous, such as in the heart 11, in the aortic arch 12, in the common carotid arteries 18/19 near the carotid sinus 20, in the subclavian arteries 13/16, or in the brachiocephalic artery 22. The baroreflex activation device 70 may be implanted such that the device 70 is positioned immediately adjacent baroreceptors 30. Alternatively, the device 70 may be positioned in the low-pressure side of the heart or vasculature, near a baroreceptor, as described in U.S. patent application Ser. No. 10/284,063, previously incorporated by reference. In fact, the baroreflex/CRT device 70 may even be positioned outside the body such that the device 70 is positioned a short distance from but proximate to baroreceptors 30. In one embodiment, the baroreflex activation device 70 is implanted near the right carotid sinus 20 and/or the left carotid sinus (near the bifurcation of the common carotid artery) and/or the aortic arch 12, where baroreceptors 30 have a significant impact on baroreflex system 50. For purposes of illustration only, an exemplary embodiment of the present invention is described with reference to the baroreflex activation device 70 positioned near the carotid sinus 20.

Memory 62 may contain data related to the sensor signal, the control signal, and/or values and commands provided by input device 64. Memory 62 may also include software containing one or more algorithms defining one or more functions or relationships between the control signal and the sensor signal. The algorithm may dictate activation or deactivation control signals depending on the sensor signal or a mathematical derivative thereof. The algorithm may dictate an activation or deactivation control signal when the sensor signal falls below a lower predetermined threshold value, rises above an upper predetermined threshold value, or when the sensor signal indicates the occurrence of a specific physiological event.

As mentioned previously, the baroreflex activation device 70 may activate baroreceptors 30 mechanically, electrically, thermally, chemically, biologically or otherwise. However, it is generally contemplated that the control signal that energizes baroreflex activation device 70 will be an electrical signal. In some instances, control system 60 includes a driver 66 to provide the desired power mode for the baroreflex activation device 70. For example, if the baroreflex activation device 70 utilizes pneumatic or hydraulic actuation, driver 66 may comprise a pressure/vacuum source and the cable 72 may comprise fluid line(s). If the baroreflex activation device 70 utilizes electrical or thermal actuation, driver 66 may comprise a power amplifier or the like and the cable 72 may comprise electrical lead(s). If baroreflex activation device 70 utilizes chemical or biological actuation, driver 66 may comprise a fluid reservoir and a pressure/vacuum source, and cable 72 may comprise fluid line(s). In other instances, driver 66 may not be necessary, particularly if processor 63 generates a sufficiently strong electrical signal for low level electrical or thermal actuation of baroreflex activation device 70.

Control system 60 may operate as a closed loop utilizing feedback from sensor 80, or as an open loop utilizing commands received by input device 64. The open loop operation of control system 60 preferably utilizes some feedback from sensor 80, but may also operate without feedback. Commands received by the input device 64 may directly influence the control signal or may alter the software and related algorithms contained in memory 62. The patient and/or treating physician may provide commands to input device 64. Display 65 may be used to view the sensor signal, control signal, and/or the software/data contained in memory 62.

The control signal generated by control system 60 may be continuous, periodic, episodic or a combination thereof, as dictated by an algorithm contained in memory 62. The algorithm contained in memory 62 defines a stimulus/activation regimen which dictates the characteristics of the control signal as a function of time, and thus dictates baroreflex activation as a function of time. Continuous control signals include a pulse, a train of pulses, a triggered pulse and a triggered train of pulses, all of which are generated continuously. Examples of periodic control signals include each of the continuous control signals described above which have a designated start time (e.g., beginning of each minute, hour or day) and a designated duration (e.g., 1 second, 1 minute, 1 hour). Examples of episodic control signals include each of the continuous control signals described above which are triggered by an episode (e.g., activation by the patient/physician, an increase in blood pressure above a certain threshold, etc.).

The stimulus/activation regimen governed by control system 60 may be selected to promote long term efficacy. It is theorized that uninterrupted or otherwise unchanging activation of baroreceptors 30 may result in the baroreceptors and/or the baroreflex system becoming less responsive over time, thereby diminishing the long-term effectiveness of the therapy. Therefore, the stimulus/activation regimen may be selected to activate, deactivate or otherwise modulate baroreflex activation device 70 in such a way that therapeutic efficacy is maintained long term. For purposes of clarity, it should be mentioned that the term stimulation and activation may be used interchangeable as well as the terms therapy and stimulus regimen. A therapy may comprise a plurality of dose regiments or regimens for delivery of different doses/intensities.

In addition to maintaining therapeutic efficacy over time, the stimulus/activation regimens of an exemplary embodiment of the present invention may be selected to reduce power requirement/consumption of control system 60. As will be described in more detail, the stimulus regimen may dictate that baroreflex activation device 70 be initially activated at a relatively higher energy and/or power level, and subsequently activated at a relatively lower energy and/or power level. The first level attains the desired initial therapeutic effect, and the second (lower) level sustains the desired therapeutic effect long term. By reducing the energy and/or power level after the desired therapeutic effect is initially attained, the power required or consumed by the device 70 is also reduced long term. This may correlate into systems having greater longevity and/or reduced size (due to reductions in the size of the power supply and associated components).

Another advantage of the stimulus/activation regimen of an exemplary embodiment of the present invention is the reduction of unwanted collateral tissue stimulation. As mentioned above, the stimulus regimen may dictate that baroreflex activation device 70 be initially activated at a relatively higher energy and/or power level to attain the desired effect, and subsequently activated at a relatively lower energy and/or power level to maintain the desired effect. By reducing the output energy and/or power level, the stimulus may not travel as far from the target site, thereby reducing the likelihood of inadvertently stimulating adjacent tissues such as muscles in the neck and head.

The stimulus/activation regimens described herein may be applied to baropacing (i.e., electrical stimulation of the carotid sinus nerve), as in the baropacing system disclosed in U.S. Pat. No. 6,073,048 to Kieval et al., the entire disclosure of which is incorporated herein by reference.

The stimulus regimen may be described in terms of the control signal and/or the output signal from baroreflex activation device 70. Generally speaking, changes in the control signal result in corresponding changes in the output of baroreflex activation device 70 which affect corresponding changes in baroreceptors 30. The correlation between changes in the control signal and changes in baroreflex activation device 70 may be proportional or disproportional, direct or indirect (inverse), or any other known or predictable mathematical relationship. For purposes of illustration only, the stimulus regimen may be described herein in such a way that assumes the output of baroreflex activation device 70 is directly proportional to the control signal. Further details of exemplary stimulus regimens may be found, for example, in U.S. Patent Application No. 60/584,730, which was previously incorporated by reference.

Control system 60 may be implanted in whole or in part. For example, the entire control system 60 may be carried externally by the patient utilizing transdermal connections to the sensor lead 82 and the control lead 72. Alternatively, control block 61 and driver 66 may be implanted with input device 64 and display 65 carried externally by the patient utilizing transdermal connections therebetween. As a further alternative, the transdermal connections may be replaced by cooperating transmitters/receivers to remotely communicate between components of control system 60 and/or sensor 80 and baroreflex activation device 70.

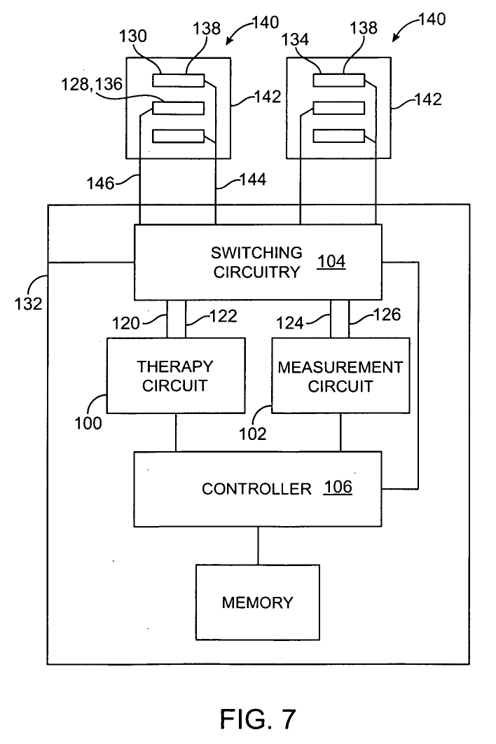

Now referring to FIG. 7, in an exemplary embodiment, a system 99 for treating a patient (not shown) is shown, including a therapy circuitry 100 for providing baroreflex activation therapy to the patient, and a measurement circuitry 102 configured for measuring a biopotential within the body of the patient for producing an electrocardiogram signal, and a switching circuit 104 coupled to the therapy circuit 100. A control circuitry/controller 106 is coupled to the switching circuitry. The control circuitry 106 is configured to direct the switching circuitry 104 to periodically connect one or more electrodes to the therapy circuit 100 for delivering baroreflex activation therapy to the patient. The control circuitry is further configured to direct the switching circuitry 104 to periodically connect the one or more electrodes to the measurement circuitry 102 to measure the biopotential within the body of the patient.

In another exemplary embodiment and as further shown in FIG. 7, the system 99 includes a therapy circuitry 100 for providing baroreflex activation therapy to a patient. Therapy circuitry 100 is connected to first and second therapy terminals 120 and 122. In the exemplary embodiment of FIG. 7, a switching circuitry 104 is also connected to first and second therapy terminals 12 and 122. The measurement circuitry 102 is connected to first and second measurement terminals, 124 and 126, and is configured for measuring a voltage between the first and the second measurement terminals, 124 and 126. The switch circuitry 104 is also connected to first and second measurement terminals 124 and 126. The switching circuitry 104 is configured to selectively couple the first and second therapy terminals, 120 and 122, to first and second electrodes 128 and 130 of an electrode assembly 140. In one example as shown, the switching circuitry selectively couples the first electrode 128 and third electrode 134, to first and second measurement terminals 124 and 126 for measuring an electric potential difference within the body of the patient.

Each of the electrodes may be part of an electrode assembly. As shown, first electrode 128 is part of an electrode assembly 140 and includes an inner electrode 136 and one or more outer electrodes 138, with the inner electrode being disposed between the two outer electrodes. The electrode assembly 140 may be formed from a sheet 142 of a flexible material, with the one or more electrodes of the electrode assembly being secured on a surface of the sheet. Each of the electrodes has a proximal end and a distal end. First and second electrical leads, 144 and 146, are electrically connected to the proximal ends of the outer electrodes 138, and the proximal end of the inner electrode 136, respectively. The inner and outer electrodes may be positioned such that opposite ends of each electrode is proximate the other. As shown, the distal end of the inner electrode 136 is positioned proximate the distal end of the outer electrodes 138.

The switching circuitry 104 may further be configured to selectively couple the first electrode 128 and a conductive housing 132, to the first and second measurement terminals 124 and 126, respectively, to enable the system to measure an electric potential within the body of the patient. In an exemplary embodiment, the conductive housing 132 may be hermatically sealed defining an interior which houses the therapy circuitry, the measurement circuitry, and the switching circuitry. The switching circuitry 104 may further be configured to selectively couple the first electrode 128 and outer electrodes 138 to the first and second therapy terminals 120 and 122, respectively, to enable the system to deliver baroreflex activation therapy to the body of the patient. The switching circuitry 104 may further be configured to selectively couple the inner electrode 136 and the outer electrodes 138 of additional electrode assemblies 140 to the first and second therapy terminals 120 and 122, respectively, to enable the system to deliver baroreflex activation therapy to additional therapy sites in the body of the patient.

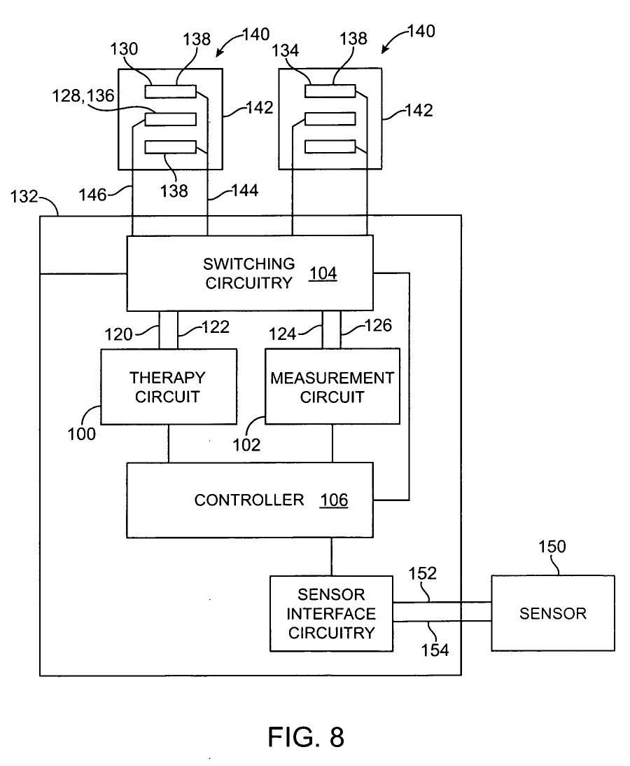

Now referring to FIG. 8, in an exemplary embodiment wherein like references refer to like elements, the baroreflex activation therapy system 99 further includes a sensor 150 connected to first and second sensor terminals 152 and 154 of a sensor interface circuitry 153. As shown in FIG. 8, sensor interface circuitry 153 is connected to controller 106. In some exemplary embodiments, sensor 150 may comprise a pressure sensor. When this is the case, sensor interface circuitry 153 may provide a pressure signal to controller 106.

For further details of exemplary baroreflex activation devices, reference may be made to: U.S. Pat. Nos. 6,522,926 and 6,616,624; and U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference.

Although the above description provides a complete and accurate representation of the invention, exemplary embodiments of the present invention may be manifested in a variety of forms other than the specific embodiments described and contemplated herein. Accordingly, departures in form and detail may be made without departing from the scope and spirit of exemplary embodiments of the present invention as described in the appended claims.

What is claimed is:

1. A method for treating a patient, comprising:
activating a baroreflex system of the patient with a baroreflex activation device according to a baroreflex activation therapy;
establishing a threshold range for a parameter indicative of a physiological condition;
monitoring the parameter; and
adjusting the baroreflex therapy in response to a value of the monitored parameter if the parameter value is outside of the threshold range.

2. The method of claim 1, wherein the threshold range is stored in a memory of the baroreflex activation device.

3. The method of claim 1, further comprising comparing a value of the monitored parameter to the threshold range of the parameter.

4. The method of claim 3, wherein the adjusting comprises discontinuing the baroreflex activation therapy if the monitored parameter is outside of the threshold range.

5. The method of claim 3, wherein the adjusting comprises discontinuing the baroreflex therapy if the value of the monitored parameter is below the parameter threshold range.

6. The method of claim 3, further comprising continuing the baroreflex therapy if the value of the monitored parameter is at or above the threshold range.

7. The method of claim 3, wherein the monitoring of the monitored parameter continues and the parameter value is compared to the threshold range to determine whether baroreflex activation therapy should continue.

8. The method of claim 5, wherein the monitoring of the monitored parameter continues and the parameter value is compared to the threshold range to determine whether baroreflex activation therapy should resume.

9. The method of claim 8, wherein the therapy is resumed once the value of the parameter is at or above the threshold range.

10. The method of claim 1, wherein the baroreflex activation therapy continues as long as the value of the monitored parameter is greater than or equal to the threshold range.

11. The method of claim 1, wherein the activation of the baroreflex system comprises activating at least one of a baroreceptor, one or more nerves emanating from a baroreceptor, and a carotid nerve.

12. The method of claim 1, wherein the activation of the baroreflex system comprises activating at least one of a baroreceptor, a mechanoreceptor, a pressoreceptor, or another receptor which affects blood pressure, nervous system activity, or neurohormonal activity in a manner analogous to baroreceptors in the arterial vasculature.

13. The method of claim 1, wherein the activation of the baroreflex system comprises activating at least one of an afferent nerve emanating from a baroreceptor, a baroreceptor, a mechanoreceptor, a pressoreceptor, or another receptor which affects blood pressure, nervous system activity, or neurohormonal activity in a manner analogous to baroreceptors in the arterial vasculature.

14. The method of claim 1, wherein the baroreflex activation therapy comprises activating a baroreceptor located in at least one of a carotid sinus, aortic arch, heart, common carotid artery, subclavian artery, and brachiocephalic artery.

15. The method of claim 1, wherein the baroreflex activation therapy comprises activating a baroreceptor located in at least one of an inferior vena cava, superior vena cava, portal vein, jugular vein, subclavian vein, iliac vein, and femoral vein.

16. The method of claim 1, wherein the baroreflex activation device is implanted in the patient.

17. The method of claim 1, wherein the activation comprises at least one of electrical activation, mechanical activation, thermal activation, chemical activation, and biological activation.

18. The method of claim 1, wherein the parameter comprises any one or more of heart rate, blood pressure, ECG, oxygen saturation, blood pH, activity level, prone posture, supine posture, core body temperature, respiration rate, respiration depth, and blood CO2 level.

19. The method of claim 1, wherein the baroreflex therapy comprises a plurality of therapy regimens at least one of which provides therapy at substantially no energy to the patient.

20. The method of claim 1, wherein the therapy regimen comprises at least one or more intensity regimens responsive to one or more characteristics of pulses generated by the baroreflex activation device.

21. The method of claim 20, wherein the pulse characteristic includes one or more of duty cycle, pulse amplitude, pulse width, pulse frequency, pulse separation, pulse waveform, pulse polarity, and pulse phase.

22. The method of claim 1, wherein the monitoring of the patient's condition comprises sensing the physiological response with one or more sensors over a period of time.

23. The method of claim 1, wherein the monitored parameter comprises heart rate.

24. The method of claim 1, wherein the monitored parameter comprises blood pressure.

25. The method of claim 22, wherein, the sensing is performed by a device comprising any one or more of extracardiac electrocardiogram, intracardiac electrocardiogram, pressure sensor, and accelerometer.

26. The method of claim 22, wherein, the activation of the baroreflex activation device is controllable by at least one of the patient and the healthcare provider.

27. The method of claim 1, wherein, the monitoring of the parameter comprises measuring at least one electrical potential difference occurring within the body.

28. The method of claim 1, wherein, the monitoring of the parameter comprises measuring a voltage difference between a first conductive element of the baroreflex activation therapy device and a second conductive element of the baroreflex activation device.

29. The method of claim 28, wherein the first conductive element comprises an electrode of the baroreflex activation device and the second conductive element comprises a conductive housing of the baroreflex activation device.

30. The method of claim 28, wherein the first conductive element comprises a first electrode of the baroreflex activation device and the second conductive element comprises a second electrode of the baroreflex activation device.

31. The method of claim 28, further comprising repeatedly measuring at least one electrical potential difference to obtain a digitized electrocardiogram waveform.

32. The method of claim 28, further identifying at least one R-wave in the electrocardiogram.

33. The method of claim 32, further comprising measuring a time interval between at least one pair of R-waves.

34. The method of claim 28, further comprising identifying at least one R-wave peak in the electrocardiogram.

35. The method of claim 34, further comprising measuring a time interval between at least one pair of R-wave peaks.

36. The method of claim 1, further comprising repeatedly measuring the value of the parameter to provide a digitized parameter waveform.

37. The method of claim 36, further comprising repeatedly measuring the value of the parameter; comparing the measured value to a second threshold value; and resuming application of baroreflex activation therapy if the measured value is greater than the second threshold value.

38. The method of claim 37, wherein the second threshold value is different than the threshold value.

39. The method of claim 37, wherein the second threshold value is greater than the threshold value.

40. A method for treating a patient, comprising:
activating a baroreflex system of the patient with a baroreflex activation device according to a baroreflex therapy comprising a plurality of baroreflex stimulation regimens wherein at least one of the regimens provides therapy at an intensity level different from another regimen;
establishing a target range for a patient parameter indicative of a physiological condition;
establishing a threshold range for the patient parameter;
monitoring the patient's parameter; and
adjusting the baroreflex therapy in response to a value of the monitored parameter if the parameter value is outside of the threshold range.

41. The method of claim 40, wherein baroreflex therapy is delivered at an initial intensity level.

42. The method of claim 40, wherein the parameter is monitored over a period of time.

43. The method of claim 40, further comprising comparing the monitored parameter to the threshold range of the parameter.

44. The method of claim 40, wherein the monitored response comprises any one or more of heart rate; blood pressure, ECG, oxygen saturation, blood pH, activity level, prone posture, supine posture, core body temperature, respiration rate, respiration depth, and blood CO2 level.

45. The method of claim 40, wherein the at least one or more intensity regimens comprises changing one or more characteristics of pulses generated to activate the baroreflex system of the patient.

46. The method of claim 40, wherein the delivery of the baroreflex therapy is by way of an open loop system, wherein the therapy is controllable by the patient or the healthcare provider.

47. The method of claim 40, wherein the delivery of the baroreflex therapy is by way of a closed loop system, wherein the therapy is controllable by a pre-programmed instructions.

48. The method of claim 40, wherein the monitoring of the patient's condition comprises sensing the parameter with one or more sensors over a period of time.

49. The method of claim 45, wherein the pulse characteristic includes one or more of duty cycle, pulse amplitude, pulse width, pulse frequency, pulse separation, pulse waveform, pulse polarity, and pulse phase.

50. The method of claim 40, wherein the monitored parameter comprises heart rate.

51. The method of claim 40, wherein the monitored parameter comprises blood pressure.

52. The method of claim 43, further comprising discontinuing the baroreflex therapy if the monitored parameter is outside the threshold range.

53. The method of claim 43, further comprising comparing the monitored parameter to the parameter target range if the monitored parameter is greater than the threshold range.

54. The method of claim 52, wherein the patient parameter is continuously monitored over a period of time and compared to the threshold range to determine whether baroreflex therapy should resume.

55. The method of claim 53, wherein if the monitored parameter is at least equal to the target range, baroreflex therapy and the monitoring of the parameter continues.

56. The method of claim 53, wherein if the monitored parameter is not equal to the target range, the baroreflex therapy is delivered according to a baroreflex activation therapy regimen responsive to the value of the monitored parameter and the monitoring of the parameter continues.

57. The method of claim 53, wherein if the monitored parameter is greater than the target range, the baroreflex therapy is delivered according to a regimen delivering baroreflex activation therapy at a higher intensity.

58. The method of claim 53, wherein if the monitored parameter is less than the target range, the baroreflex therapy is delivered according to a regimen delivering therapy at a lower intensity.

59. A system for treating a patient, comprising:
a therapy circuitry for delivering baroreflex activation therapy to the patient;
a controller circuitry configured for applying the baroreflex activation therapy to the patient, the controller connectable to the therapy circuitry; and
a memory circuitry in communication with the controller and configured for storing information regarding the baroreflex activation therapy.

60. The system of claim 59, wherein the therapy circuitry comprises a pulse generator configured for generating stimulation pulses to activate the baroreflex system of the patient, wherein the pulse generator is configured for delivery of a plurality pulses having different intensity levels.

61. The system of claim 59, wherein the baroreflex activation therapy comprises a plurality of therapy regimens comprising different intensity levels, wherein at least one of the intensity levels is at or close to zero.

62. The system of claim 60, wherein the baroreflex activation therapy includes a plurality therapy regimens at least one of which is different than another regimen.

63. The system of claim 60, wherein the system further comprises at least one electrode assembly.

64. The system of claim 63, wherein the electrode assembly is locatable proximate one or more baroreceptors of the patient.

65. The system of claim 59, wherein the system further comprises a monitoring circuitry connectable to the controller circuitry.

66. The system of system 65, wherein the system further comprises a sensor connectable to the monitoring circuitry and which is configured for sensing of the patient parameter which is indicative of a physiological condition.

67. The system of claim 65, wherein the sensor comprises one or more of extracardiac electrocardiogram, intracardiac electrocardiogram, pressure sensor, and accelerometer.

68. The system of system 66, wherein the controller circuitry is configured to adjust the baroreflex activation therapy based on information received by way of the sensor.

69. The system of claim 65, further comprising a switching circuitry connectable to the monitoring circuitry and the therapy circuitry for adjusting the baroreflex activation therapy based on the information received from the monitoring circuitry and the therapy circuitry.

70. The system of claim 69, wherein the switching circuitry is connectable to at least one electrode assembly locatable proximate one or more baroreceptors of the patient.

71. The system of claim 59, wherein the system is housed within a single housing.

72. The system of claim 59, wherein the system is implantable in the patient.

73. The system of claim 59, wherein the system is further configured for communication with other devices comprising: cardiac rhythm management devices comprising cardiac resynchronization therapy (“CRT”) devices, cardioverters, defibrillators, pacemakers, and combinations thereof.

74. A system for treating a patient, comprising:
a therapy circuitry for providing baroreflex activation therapy (BAT) to a body of a patient;
a monitoring circuitry that is capable of measuring a biopotential within the body of the patent for producing an electrocardiogram signal;
a switching circuitry coupled to the therapy circuitry and the measurement circuitry; and
a control circuitry coupled to the switching circuitry, the control circuitry configured for directing the switching circuitry to periodically connect one or more electrodes to the therapy circuitry for providing baroreflex activation therapy (BAT) to the body of the patient, and the control circuitry configured for directing the switching circuitry to periodically connect the one or more electrodes to the monitoring circuitry for measuring the biopotential within the body of the patent for producing the electrocardiogram signal.

75. A baroreflex activation therapy (BAT) system, comprising:
a therapy circuitry connected to a first therapy terminal and a second therapy terminal for producing a baroreflex activation therapy signal;
a measurement circuitry that is capable of measuring a voltage between a first measurement terminal and a second terminal; and
a switching circuitry connected to the first measurement terminal, the second terminal, the first therapy terminal, and the second therapy terminal; the switching circuitry selectively coupling a first electrode and a second electrode to the first therapy terminal and the second therapy terminal, respectively, for providing baroreflex activation therapy to a body of a patient.

76. The system of claim 75, wherein the switching circuitry selectively couples the first electrode and a conductive housing of the BAT system to the first measurement terminal and the second measurement terminal, respectively, for measuring an electric potential difference within the body of the patient.

77. The system of claim 76, wherein the conductive housing comprises a hermetically sealed housing defining an interior.

78. The system of claim 77, wherein the therapy circuitry, the measurement circuitry, and the switching circuitry are all disposed in the interior of the housing.

79. The system of claim 75, wherein the switching circuitry selectively couples the first electrode and a third electrode to the first measurement terminal and the second measurement terminal, respectively, for measuring an electric potential difference within the body of the patient.

80. The system of claim 75, wherein the first electrode comprises an inner electrode of a BAT electrode assembly.

81. The system of claim 75, wherein the first electrode comprises an outer electrode of a BAT electrode assembly.

82. The system of claim 81, wherein the BAT electrode assembly comprises a sheet of flexible material and a plurality of electrodes secured over a first surface of the sheet.

83. The system of claim 82, wherein the plurality of electrodes comprises:
an inner electrode, a first outer electrode, and a second outer electrode, wherein
each electrode has a proximal end and a distal end, and wherein the inner electrode is located between the first outer electrode and the second outer electrode;
a first lead electrically connected to the proximal ends of the first outer electrode and the second outer electrode; and
a second lead electrically connected to the proximal end of the inner electrode.

84. The system of claim 83, wherein the plurality of electrodes are positioned on the sheet such that the proximal end of the first outer electrode and the distal end of the inner electrode are positioned proximate one another; and the distal end of the first outer electrode and the proximal end of the inner electrode are positioned proximate one another.

85. A baroreflex activation therapy device, comprising:
a memory storing a threshold value associated with a physiological parameter of a patient;
a therapy circuitry for delivering baroreflex activation therapy to a body of the patient;
a sensor for measuring a value of a physiological parameter of the patient; and
a disable circuitry that disconnects the therapy circuitry from at least one patient electrode if the measured value is below the threshold value.

86. The system of claim 85, wherein the sensor comprises a pressure sensor.

87. The system of claim 85, wherein the sensor comprises a measurement circuitry connected to one or more electrodes for measuring a biopotential in the body of the patient.

88. A baroreflex activation therapy (BAT) system, comprising:
a therapy circuitry connected to a first therapy terminal and a second therapy terminal for producing a baroreflex activation therapy signal;
a controller connected to the therapy circuitry;
a sensor connected to the controller;
a switching circuitry connected to the controller; and
the switching circuitry being connected to the first sensor terminal and the second terminal, wherein the switching circuitry selectively couples a first electrode and a second electrode to the first therapy terminal and the second therapy terminal, respectively, for providing baroreflex activation therapy to a body of a patient.

89. The system of claim 88, wherein the sensor comprises a pressure sensor.
